(19) United States
(12) Patent Application Publication
(10) Pub. No.: US 2007/0156077 A1
(43) Pub. Date: Jul. 5, 2007

(54) Personal hygiene system and methods

(76) Inventor: Margaret M. Pfister, Hinsdale, IL (US)

Correspondence Address: Charles C. Valauskas;BANIAK PINE & GANNON, Suite 1200, 150 N. Wacker Drive, Chicago, IL, 60606, US

(21) Appl. No.: 11/323,869

(22) Filed: Dec. 30, 2005

(51) Int. Cl. A61M 35/00

(52) U.S. Cl. 604/2; 604/289

(57) ABSTRACT

A system and methods for treating a human subject surface is provided. The system includes an applicator device for promoting hygiene, which includes a handle including a first handle end sized and shaped to be held by a user and a second handle end opposite the first handle end. An applicator includes a body formed of hygienic material, wherein the applicator body includes a proximal applicator end attached to the second handle end and a distal applicator end opposite the proximal applicator end. The distal applicator end is sized and shaped to be usable in transferring a fluid to a part of the human anatomy. The invention provides the applicator device and fluid in kit form and methods of use.

### FIELD OF THE INVENTION

The present invention relates to a system and methods for the administration of personal hygiene, e.g., cleansing, treatment and therapy. More particularly, the present invention relates to a system and methods by which a user may manually apply a fluid for the purpose of maintaining or achieving personal hygiene. Certain preferred embodiments are related to maintaining or achieving internal personal hygiene while others may be used for external hygiene purposes and may be applied to either or both of females or males.

Specifically, certain embodiments of the invention include an applicator device that includes a handle and an attached applicator tip, by which a fluid may be applied to an internal or external surface of a human subject. The fluid may be of a composition to achieve gentle cleansing of the desires surface, or reestablish or maintain a desired chemical and/or biological condition in, on, or around the subject surface, or to alter the chemical and/or biological condition of the subject surface. The tip may be adapted to complement the purpose of, or aid in the delivery of the fluids by being sized and shaped to apply the fluid to the subject area to be cleansed or treated. The invention contemplates various sizes and shapes of applicator tip, depending upon the particular intended use. The invention also contemplates providing the applicator device in kit form, i.e., in combination with various fluids and associated containers directed to various cleansing and treatment needs.

### BACKGROUND OF THE INVENTION

At present, numerous products are available for hygienic purposes, both internal and external. When designed for internal use with a female subject, these prior art hygienic devices may take the form of a plastic container that when squeezed, inserts a liquid into the vagina. Other devices resemble a pad or tampon. A number of devices have been proposed for the insertion of feminine hygienic devices and the promotion of good hygiene. Sterile cloth pads are most commonly used in cleansing, typically with a disinfectant, like soap, iodine, alcohol and other compounds in solution. Other devices for transfer of an active liquid into an intracorporeal cavity are complicated, at least in some cases, because they are designed to both deliver and recover the active liquid. In summary, many of these devices are either not easy or safe to use when intravaginal cleansing is desired and other devices are unnecessarily complex.

Accordingly, there is a demand therefore for simple, safe, and effective devices, systems and methods for use in hygienic cleansing and treatment of human males and females. The present invention satisfies the demand.

### SUMMARY OF THE INVENTION

The present invention contemplates a system and methods for maintaining, reestablishing and/or altering the physical, chemical, and/or biological condition on or around the target area of females and/or males. The system and methods may be used internally and/or externally.

In broad form, the present invention includes a system including an applicator and handle. The system may include one or more fluid for use with the applicator and handle. The applicator and handle may include a fluid delivery system. The fluid delivery system may include a hollow handle, which functions as a reservoir for the fluid, and a plunger or other mechanism to dispense the fluid from the reservoir. The applicator and handle may be provided without a fluid delivery system.

The present invention may be provided in a combined or multiple form with a variety of sizes and shapes of applicator tips. The combined form may include one or more fluid. The combination may be a kit. The kit may be packaged together or may be separately provided.

One example of the system of the present invention includes an applicator device, which when used for internal cleansing of a female subject, and includes an applicator tip, which may be of an absorbent material, to deliver a liquid inside the subject's vagina. This device can then be manipulated to provide more thorough cleansing of the vagina.

The applicator device may be optionally provided and/or packaged with a tray for holding a fluid and conveying the fluid to the applicator device. The applicator device may be sealed in a package in a dry/sterile condition. Alternately, the applicator device may be sealed in a package in a sterile condition with the appropriate fluid pre-applied thereto, or a “wet” condition.

Another embodiment of the invention provides a hygienic device, and includes a handle including a first handle end sized and shaped to be held by a user and a second handle end opposite the first handle end. An applicator includes a body formed of an absorbent material, wherein the applicator body includes a proximal applicator end attached to the second handle end and a distal applicator end opposite the proximal applicator end. The distal applicator end is sized and shaped to be usable in transferring a fluid to a part of the human anatomy Additional embodiments of the system include an applicator with which different materials may be used for cleansing and/or prevention of conditions that may require medical treatment. Such medical conditions include vaginal infections, specifically yeast infections and bacterial vaginosis. Additionally, the present invention may include an applicator and materials for the delivery of medication to a treatment area. Among the types of medication that could be delivered is that which treats bacterial inflammation from bacterial vaginosis and the inflammation that occurs from retained foreign bodies. Furthermore, the system of the present invention can be used for external cleansing such as in the vulva, perineum and rectal area of females and the penis, scrotum and rectal areas of males.

Examples of fluids useful in the present invention include cleansing fluids, e.g., sterile water and other aqueous based solutions, therapeutic agents for use in treating and/or preventing infections and non-infection diseases or conditions, prophylactic agents, diagnostic agents, immuno-therapeutic agents, cosmetic or personal hygiene agents, antiseptic agents, bactericidal agents, fungicidal agents, spermicidal agents, local treatment agents, systemic agents, trophic agents and lubricant agents.

With regard to feminine internal cleansing, the present invention includes an applicator that delivers a fluid by means of an applicator tip, which is then manually manipulated for internal vaginal cleansing. The applicator tip may be an absorbent and/or spongy material. The applicator tip is combined with a 6-inch handle so the user may manipulate the device from outside the body.

The applicator tip can have different shapes including rectangle, rectangle with a concave or convex semi-circular distal end, square and conical, for example. Additionally, a small size of each of these tip shapes is contemplated to accommodate nulliparous women.

The range of uses of this invention is further broadened by the different solutions that may be packaged with the disposable applicator. In one example, sterile water could be packaged with the intention of using the sterile water and applicator for gentle internal cleansing. In another example, the applicator could be packaged with a 5% acetic acid solution for restoration of the normal pH of the vagina. In yet another example, the applicator could be packaged with a 1% chlorhexidine gluconate solution for more extensive internal cleansing and the reduction or elimination of bacteria from the vagina.

The packaging of the solutions can be provided in at least two different ways. In one example, a plastic bottle and a molded plastic tray could be combined with the applicator device such that the liquid solution would be poured into the tray so that the applicator tip could be placed in the plastic tray to absorb the solution. Once the solution is absorbed, the device can then be used for its intended purpose. In another example, a pouch or package of leak proof material, e.g., aluminum foil is provided with an effective amount of treatment fluid, and one or more applicator devices. The package would only need to be opened to be used.

With respect to external hygienic cleansing, the applicator could include the same 6-inch handle to which is fixed an applicator tip adapted for use with different shapes as detailed above. For external cleansing purposes, the applicator tip could be packaged with one or more fluids or solutions, e.g., sterile water and/or 1% chlorhexidine gluconate. The device could be packaged as above with either a plastic bottle filled with the appropriate solution and a molded plastic tray or in pre-saturated aluminum foil bags.

The invention contemplates various methods of use, related to releasing the applicator device from a package, grasping the applicator device by a handle portion thereof, conveying a fluid to an applicator tip of the applicator device, contacting a subject surface with the applicator tip and manipulating the applicator device to provide one or more of cleaning and treating the subject surface.

These and other advantages, as well as the invention itself, will become apparent in the details of construction and operation as more fully described and claimed below.

### BRIEF DESCRIPTION OF THE DRAWINGS

FIG. 1 is a perspective view of a device according to one embodiment of the invention;

FIG. 2 is a perspective view of a device according to a second embodiment of the invention;

FIG. 3 is a perspective view of a device according to a third embodiment of the invention;

FIG. 4 is a perspective view of a device according to a fourth embodiment of the invention;

FIG. 5 is a perspective view of a device according to a fifth embodiment of the invention;

FIG. 6 is a perspective view of a device and kit according to an embodiment of the invention;

FIG. 7 is a perspective view of a device and kit according to an alternate embodiment of the invention;

FIG. 8A is a perspective view of a device according to an additional embodiment of the invention; and

FIG. 8B is a perspective view of a device according to an alternate embodiment of the invention.

### DETAILED DESCRIPTION OF EMBODIMENTS OF THE INVENTION

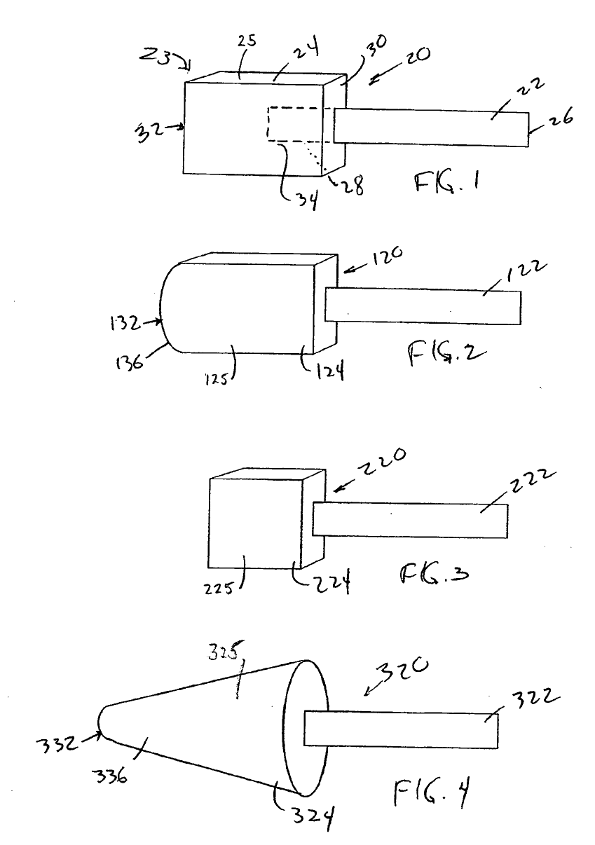

FIG. 1 shows an embodiment of a system for providing personal hygiene according to the invention including an applicator device 20 in one form thereof. Generally, the applicator device 20 includes a handle 22 with an attached body 23. The body 23 forms an applicator tip 24.

The handle 22 is sized and shaped to be usable both for grasping by the user and for attaching the applicator tip 24. The handle 22 in the illustrated embodiment has a flat rectangular shape and is about six inches in length. It will be understood that the handle 22 may be any suitable length, according to the intended use of the device 20, the material used and other variables, for example, and is preferably of a length to permit manipulation of the applicator tip 24 when the tip 24 has been inserted internally into the subject. The handle 22 may be made in a variety of shapes and sizes to facilitate the manipulation of the device 20. For example, the handle 22 may be solid or hollow, rectangular, cylindrical, triangular, pentagonal, or hexagonal (not shown).

The handle 22 may be made of any handle material to facilitate the manipulation of the device 20. Examples of suitable handle material include synthetic and natural materials, including paper, wood, plastic, metal, and composites.

Certain embodiments of the handle 22 are sized and shaped to be graspable by the user and includes a first handle end 26 and, opposite thereto, a second handle end 28, affixed within, adhered or attached or fastened to or releasably disposed within or operatively associated with the applicator tip 24 by suitable means or method.

The applicator tip 24 shown is rectangular in overall morphology and includes a proximal applicator end 30, at which the handle 22 is attached and a distal applicator end 32, opposite the proximal end 30. Other applicator tip morphologies are contemplated by the invention, some of which will be discussed in more detail herein, and are sized and shaped at least to be adapted for different applications in use in cleaning and other hygienic activities.

The handle 22 may be received in the proximal applicator end 30, by fitting inside of a socket 34, which may be in the form of a hole or slit, or the like, in the material of the applicator proximal end 30. The handle 22 may be held in the socket 34 by friction, an adhesive (not shown), a co-molding process, heating one or both of the handle 22 and applicator tip 24, or any other suitable means or method.

The applicator tip 24 may be made of any suitable material to permit the administration of personal hygiene. Examples of suitable material for the applicator 24 include natural and artificial sponge, manufactured foam, paper, biocompatible materials, and composites of same. These materials will be collectively identified also as “hygienic material” and identified by the number 25.

In certain embodiments, the hygienic material 25 of the applicator tip 24 is designed to be absorbent and hold and dispense or apply a fluid, solution, or the like, in use. Examples of fluids intended to be used with the applicator device 20 of the present invention include water, sterile water, acetic acid solution (e.g., 5%), and a chlorhexidine gluconate (1%). Other fluids, medicaments, solutions and so on are contemplated by the invention as will be understood by those familiar with the present subject.

The fluids usable with the applicator device 20 of the present invention are intended to function in the cleaning, disinfection and treatment of various conditions and parts of the body. For example, the applicator device 20 is designed to be used both internally and externally. In one embodiment, the applicator device 20 is usable for internal cleansing of the vagina by both delivering a cleansing fluid and for physical cleansing by manually manipulating the applicator device. Furthermore, the device 20 may be used externally for cleansing and so on, by contacting the vulva, perineum and rectal areas of females and the penis, scrotum and rectal areas of males. The device 20 may include types of fluid generally directed to gentle cleansing, the prevention of conditions, and the medication of conditions. Use of the present invention is not necessarily limited to genital areas of males and females.

With regard to the methods of feminine internal cleansing or the cleansing or treatment of a subject (human) surface according to embodiments of the invention, the device 20 of the present invention may be first released from a sterile packaging (not shown, but well known). A fluid is selected according to the condition being treated, related to cleansing or treatment of the condition. The selected fluid is applied the applicator tip 24, by any suitable method. The user grasps the handle 22 and contacts the wetted applicator tip 24 to a subject surface. The user manipulates the handle 22, and thus the applicator device 20 over the subject surface to promote both transfer of the fluid to the surface, absorption of fluid from the surface, and alternately, physical cleansing, e.g., debridement of the surface.

In alternate embodiments, the fluid may be pre-existing in the material 25 of the applicator tip or may be absorbed therein or applied thereto just before use.

Other methods of use will be described in more detail hereinbelow. These methods may be used with the depicted systems regardless of size and shape of the devices and regardless of the subject surface to be treated and so may apply to all of the devices shown in FIGS. 2-7.

FIG. 2 shows another device according to an embodiment of the invention is shown generally at 120. The device 120 is similar to that of FIG. 1, including a handle 122 and applicator tip 124 formed from hygienic material 125. In particular, the applicator tip 124 includes a distal end 132, which includes a convex surface 136. The convex surface 136 may be more suited to reaching and/or conforming to an inter-vaginal area.

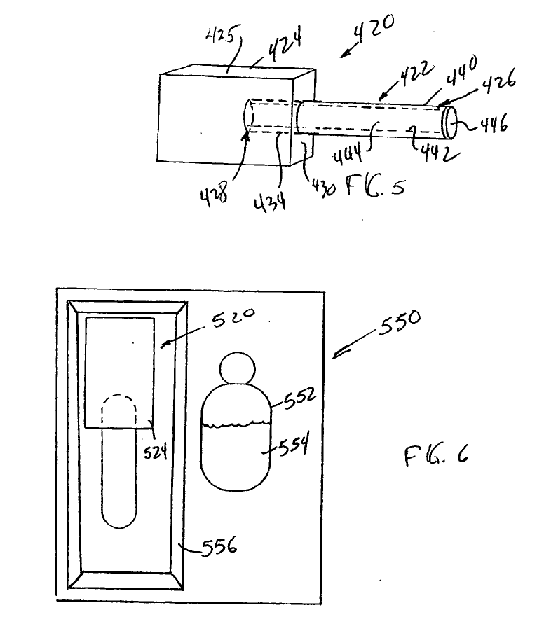

FIG. 3 illustrates another device according to an embodiment of the invention generally at 220. The device 220 is similar to that of FIG. 1, including a handle 222 and applicator tip 224 formed from hygienic material 225. In particular, the applicator tip 124 includes an overall square shape as opposed to the generally rectangular shape of the device shown in FIG. 1. The square shape of the applicator tip 224 may be more suited to reaching and/or conforming to certain areas intended to be contacted by the device 220.

FIG. 4 illustrates another device according to an embodiment of the invention generally at 320. The device 320 is similar to that of FIG. 1, including a handle 322 and applicator tip 324 formed from hygienic material 325. In particular, the applicator tip 324 includes a distal end 332, which includes a conical surface 336. The conical surface 336 may be more suited to reaching and/or conforming to the area intended to be contacted by the device 320. It will be understood that the applicator tip 324 of the device 320 may be formed into any suitable shape desired.

For certain applications, the devices (20, 120, 220, 320) shown in FIGS. 1-4 may range in size from about two inches to about ±2 inches in length, from about 1 inch to about ½ inches in width, and about one half inch in depth. Other sizes are contemplated dependent upon intended use and other considerations.

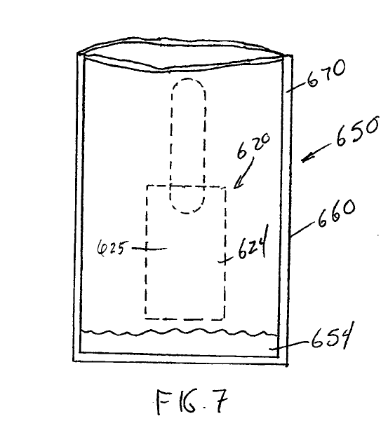

FIG. 5 illustrates yet another applicator device 420 according to an embodiment of the invention. In a most general form, the applicator device 420 includes a handle 422 with an attached applicator tip 424 formed from hygienic material 425.

The handle 422 is sized and shaped to be usable both for grasping by the user and for attaching to the applicator tip 424. The handle 422 in the illustrated embodiment has a hollow cylindrical shape and that may be about six inches in length. The handle 422 includes a cylindrical outer surface 440 and a cylindrical internal surface 442, which defines a cylindrical internal handle chamber 444.

It will be understood that the handle 422 may be any suitable length, according to the intended use of the device 420, the material used and other variables, for example, and is preferably of a length to permit manipulation of the applicator tip 424 when the tip has been inserted internally into the subject, for example. The handle 422 may be made in a variety of shapes and sizes. For example, the handle 422 may be rectangular, cylindrical, triangular, pentagonal, hexagonal and so on (not shown). The handle 422 may be made of any suitable handle material 425 as discussed above.

The handle 422 has a first handle end 426, which is sized and shaped to permit manipulation by the user. At the first handle end 426 is a cap 446, or the like, which may be releasably attached to the handle first end 426 to close the internal handle chamber 444. The handle 422 has a second handle end 428, opposite the first handle end 426, which is attached to the applicator tip 424. The second handle end 428 may be affixed within the applicator tip 424, adhered or fastened to, releasably disposed within or operatively associated with the applicator tip by any other suitable means or method. In an alternate embodiment, the chamber 444 may be provided with a syringe (not shown) for actively promoting migration of the fluid from the chamber to the applicator 424.

The handle 422 may be received in a proximal applicator end 430, by fitting inside of a socket 434, which may be in the form of a hole or slit, or the like, in the material of the applicator proximal end 430. The handle 422 may be held in the socket 434 by friction, an adhesive (not shown), a co-molding process, heating one or both of the handle 422 and applicator tip 424, or any other suitable means or method.

The hygienic material 425 of the applicator tip 424 is designed to be absorbent and hold and dispense or apply a fluid, solution, or the like, in use. Examples of fluids intended to be used with the applicator device 420 of the present invention are discussed above. The embodiment of the device 420 shown in FIG. 5 contemplates use of the internal handle chamber 444 as a reservoir for the fluids (not shown). In one example, the handle 422 may be formed of a flexible material, so that the user may squeeze fluid from the internal handle reservoir 444 into the material of the applicator tip 424. In other examples, fluid may enter the material of the applicator tip 424 by wicking or other known physical or mechanical means.

With regard to the methods of internal or external cleansing or the cleansing or treatment of a subject (human) surface according to embodiments of the invention, the device 420 of the present invention may be first released from a sterile packaging (not shown, but well known). The type of fluid in the chamber 444 is selected according to the condition being treated, related to cleansing or treatment of the condition. The fluid is urged into the applicator tip 424, by a fluid delivery mechanism. In one example, actuation of the fluid delivery mechanism includes manipulating the handle 422 to urge fluid from the chamber 444, into the material of the applicator tip 424. An alternate embodiment contemplates removing the cap 446, inserting a plunger (not shown, but well known) and operating the plunger to urge the fluid from the chamber 444 wetting the applicator tip 424. The user then grasps the handle 422 and contacts the wetted applicator tip 424 to a subject surface. The user manipulates the handle 422, and thus the applicator device 420 over the subject surface to promote both transfer of the fluid to the surface, absorption of fluid from the surface, and alternately, physical cleansing, e.g., debridement of the surface.

FIG. 6 shows another embodiment of the invention wherein a device according to FIGS. 1-5 may be combined with other devices to form a kit 550. The kit 550 may include a container 552 of fluid 554 and a tray 556, sized and shaped to receive the fluid 554 and permit absorption of the fluid by the applicator device 520. In use, the fluid 554 is dispensed from the container 552 into the tray 556. An applicator tip 524 of applicator device 520 is inserted into the tray 556 to permit some or all of the fluid 554 to be absorbed or transferred to the applicator tip. The applicator device 520 may then be used to contact and treat the subject.

FIG. 7 shows another embodiment of the invention wherein a device according to FIGS. 1-5 may be provided in a pre-packaged kit 650. The kit 650 may include a packet 660 in the form of a fluid tight pouch, or the like, containing an effective amount of fluid 654. The pouch 660 may be provided in a sealed condition with a peripheral seal 670. The pouch 660 is sized and shaped to receive the fluid 654 and an applicator device 620. In use, the pouch 660 is opened and the applicator device 620 is withdrawn therefrom. The fluid 654 is pre-absorbed into or pre-wetted onto the hygienic material 625 of an applicator tip 624 of the device 620. The applicator device 620 may then be used to contact and treat the subject.

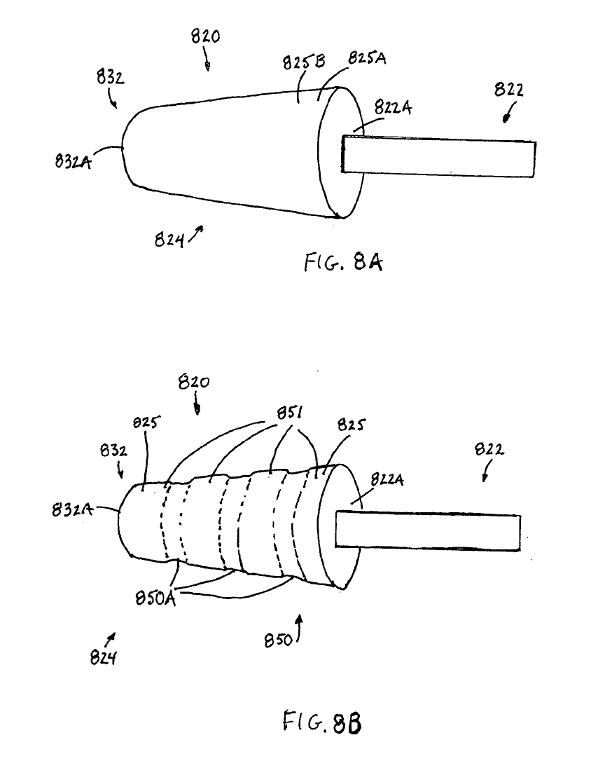

FIG. 8A and FIG. 8B show additional embodiments of an applicator device 820. The device 820 is similar to that shown in the other Figures and includes a handle 822 and an applicator tip 824 formed from hygienic material 825. The applicator tip 824 includes a surface 825A having a rounded conical shaped formed by a wall 825B that converges from the area 822A at which the handle 822 makes contact with the applicator tip 824 toward a distal end 832. The illustrated embodiment of the applicator device 820 includes a broad and rounded surface 832A.

The applicator device 820 may include one or more applicator elements to facilitate the cleansing or delivery of medication. FIG. 8B includes applicator elements 850 for such purposes. The applicator elements 850 are sized and shaped as indentations 850A, thereby providing broad raised areas 851 in the applicator tip 824.

Whilst endeavoring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon. While the apparatus and method herein disclosed forms a preferred embodiment of this invention, this invention is not limited to that specific apparatus and method, and changes can be made therein without departing from the scope of this invention, which is defined in the appended claims.

What is claimed is:

1. A system for treating a subject surface, comprising:
a handle including a first handle end sized and shaped to be held by a user and a second handle end opposite said first handle end; and
an applicator including a body formed of hygienic material, said applicator body including a proximal applicator end attached to said second handle end and a distal applicator end opposite said proximal applicator end, said distal applicator end being sized and shaped to transfer fluid to a part of the subject surface.

2. The system of claim 1, wherein said handle is a flattened rectangle in shape.

3. The system of claim 1, wherein said handle is hollow and includes a chamber formed therein.

4. The system of claim 3, wherein said internal chamber is defined, at least in part, by an internal surface of said handle.

5. The system of claim 4, wherein said internal surface is generally cylindrical.

6. The system of claim 3, wherein said first handle end includes a removable cap that closes a first end of said chamber.

7. The system of claim 3, wherein said chamber is adapted to hold and dispense a fluid.

8. The system of claim 7, wherein said handle is formed of a material permitting said handle to be manipulated to cause said fluid to be transferred to said applicator from said chamber.

9. The system of claim 7, including a mechanism for urging said fluid from said chamber to said applicator.

10. The system of claim 1, wherein said applicator body is rectangular.

11. The system of claim 1, wherein said applicator body is square.

12. The system of claim 1, wherein said applicator body is cone shaped.

13. The system of claim 1, wherein said applicator body is cylindrical.

14. The system of claim 1, wherein said distal applicator end includes a convex surface.

15. The system of claim 1, further including a fluid.

16. The system of claim 15, wherein said fluid is water.

17. The system of claim 15, wherein said fluid is sterile water.

18. The system of claim 15, wherein said fluid is a cleansing fluid.

19. The system of claim 15, wherein said fluid is a disinfectant.

20. The system of claim 15, wherein said fluid is a medicament.

21. The system of claim 15, wherein said fluid is a 1% chlorhexidine gluconate solution.

22. The system of claim 15, wherein said fluid is a 5% acetic acid solution.

23. The system of claim 15, wherein said fluid is one or more of a therapeutic agent for use in treating and/or preventing infections and non-infection diseases or conditions, a prophylactic agent, a diagnostic agent, an immuno-therapeutic agent, a cosmetic agent, a personal hygiene agent, an antiseptic agent, a bactericidal agent, a fungicidal agent, a spermicidal agent, a local treatment agent, a systemic agent, a trophic agent and a lubricant.

24. A system for treating a subject surface, comprising:
a handle including a first handle end sized and shaped to be held by a user and a second handle end opposite said first handle end;
an applicator including a body formed of hygienic material, said body including a proximal applicator end disposed on said second handle end and a distal applicator end opposite said proximal applicator end, said distal applicator end being sized and shaped to be usable in contacting the subject surface; and
a fluid for the treatment of the subject surface.

25. The system of claim 23, wherein said fluid is pre-existing on said applicator.

26. The system of claim 24, wherein said fluid is applied to said applicator just prior to contacting the subject surface.

27. The system of claim 24, further including a tray sized and shaped to hold said fluid and receive said distal applicator end to transfer said fluid thereto.

28. The system of claim 24, further including a container for containing said fluid prior to transferring said fluid to a part of the subject surface.

29. The system of claim 24, further including a pouch sized and shaped to receive said fluid, said handle and said applicator.

30. The system of claim 24, further including a delivery mechanism for said fluid.

31. A system for treating a subject surface with a fluid, in combination, comprising:
an applicator device including a handle including a first handle end sized and shaped to be held by a user and a second handle end opposite said first handle end and an applicator including a body formed of hygienic material, said applicator body including a proximal applicator end attached to said second handle end and a distal applicator end opposite said proximal applicator end, said distal applicator end being sized and shaped to be usable in transferring the fluid to the subject surface;
a container sized to contain said fluid, said fluid for one or both of cleansing and treating a the subject surface; and
a tray sized and shaped to receive said fluid from said container and transfer said fluid to said applicator device.

32. A system for treating a subject surface with a fluid, in combination, comprising:
an applicator device including a handle including a first handle end sized and shaped to be held by a user and a second handle end opposite said first handle end and an applicator including a body formed of hygienic material, said applicator body including a proximal applicator end attached to said second handle end and a distal applicator end opposite said proximal applicator end, said distal applicator end being sized and shaped to be usable in transferring a fluid to the subject surface;
a pouch sized and shaped to contain said fluid, said fluid for one or both of cleansing and treating a part of the human anatomy and said applicator device.
